(19) United States
(12) Patent Application Publication
(10) Pub. No.: US 2009/0317371 A1
(43) Pub. Date: Dec. 24, 2009

(54) Method, Composition, and Device, for the Treatment of Amylase Malfunctions / Inactivity in Association with Saccharides (Mainly Polysaccharides) Based Diseases

(75) Inventor: Tarig Sayed Mustafa Arbab, London (GB)

Correspondence Address: Tarig Sayed Mustafa Arbab, 13 Argyle Road, North Finchley, London, N12 8JA, GB

(73) Assignee: Saccharides Science & Technology Ltd., London (GB)

(21) Appl. No.: 12/165,378

(22) Filed: Jun. 30, 2008

(30) Foreign Application Priority Data
Jun. 19, 2008 (GB) GB0811274.0

(51) Int. Cl. A61K 38/54; A61P 1/14; C12Q 1/54

(52) U.S. Cl. 424/94.2; 435/14

(57) ABSTRACT

Physiological protein and enzyme complex having active Trimethylglycine, active amylases, active lipases, and active proteases, of human (saliva enzymes), yeast, plant, fungi, and or microbial origin, preferably digestive enzyme complex such as, Trimethylglycine, 4-α-D-glucan glucanohydrolase; Exo-1,4-α-glucosidase; Beta-fructofuranosidase; Protease (3.0); Pectinase; Lipase; Cellulase; Lactase; Malt Diastase, or digestive enzyme complex containing proteins and enzymes for the treatment of high levels of saccharides and correction of amylases malfunctioning activity or inactivity in the body. The invention also relates to the production of pharmaceutical compositions suitable for such treatment. A preferred variant of the invention relates to use of this enzyme complex having Trimethylglycine, amylolytic, lipolytic, and proteolytic activity, especially salivary, gastric, pancreatic and intestinal enzyme complex for the treatment of malfunctioning, or absent amylases enzymes activity but not deficiency, in association with or without excess saccharides in the body.

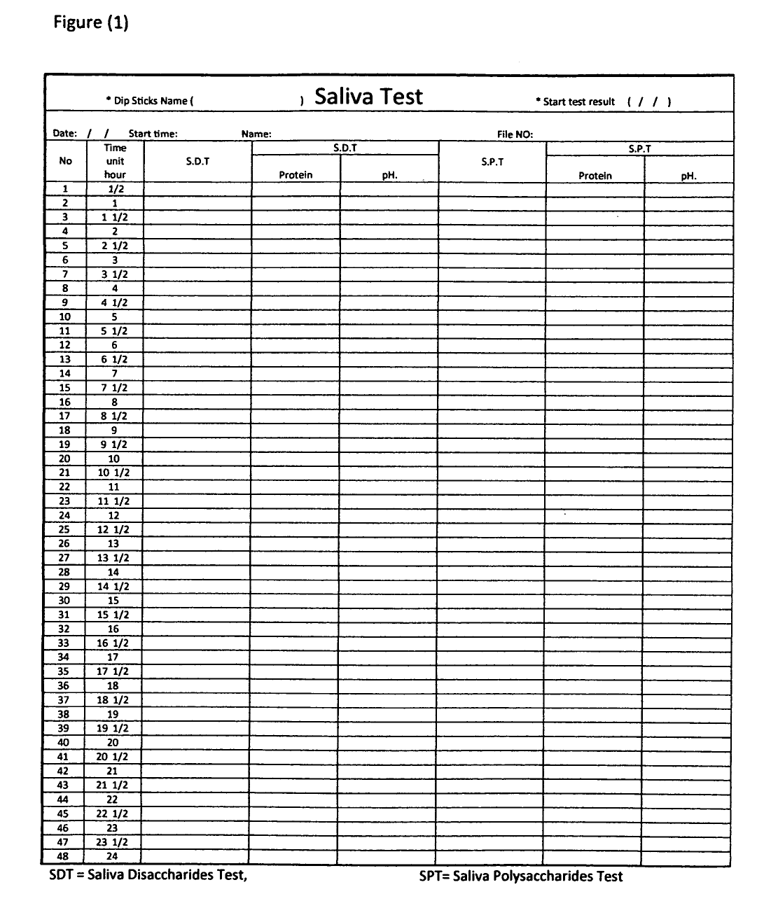

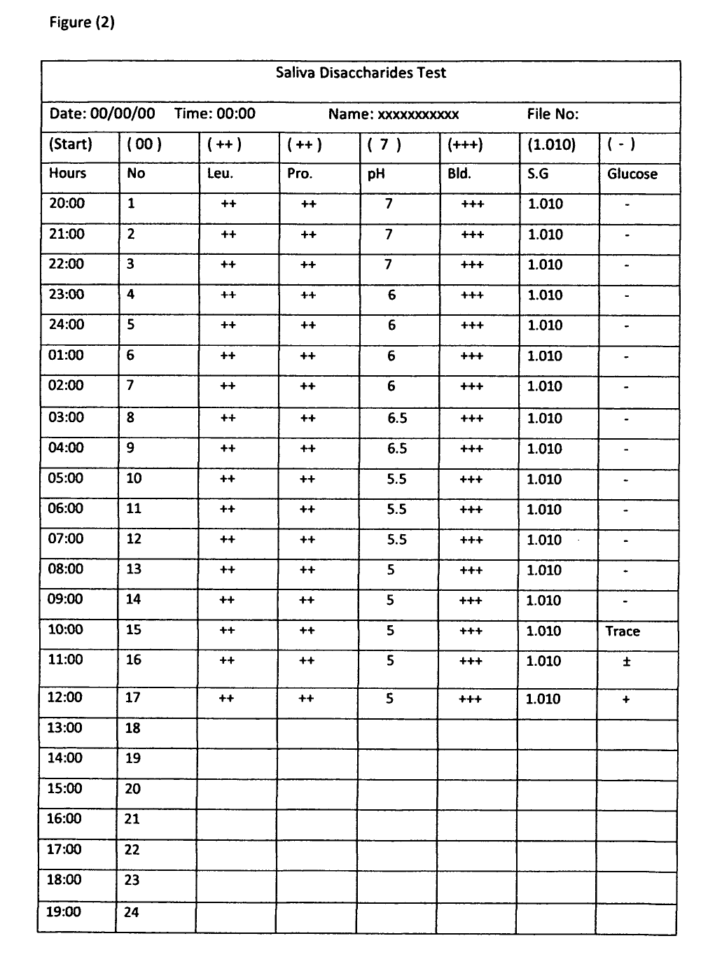

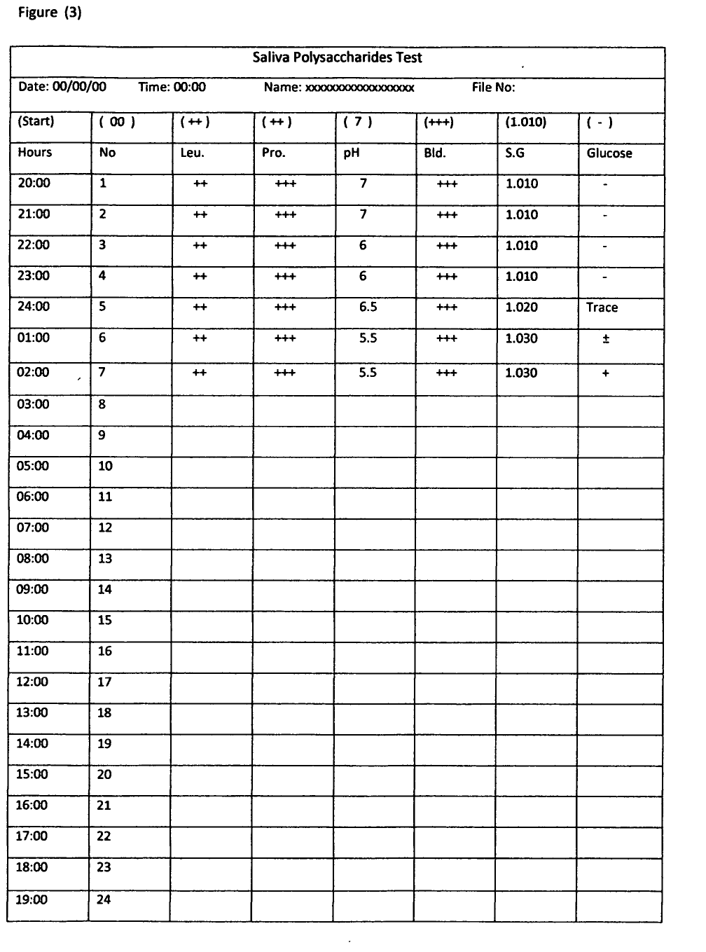

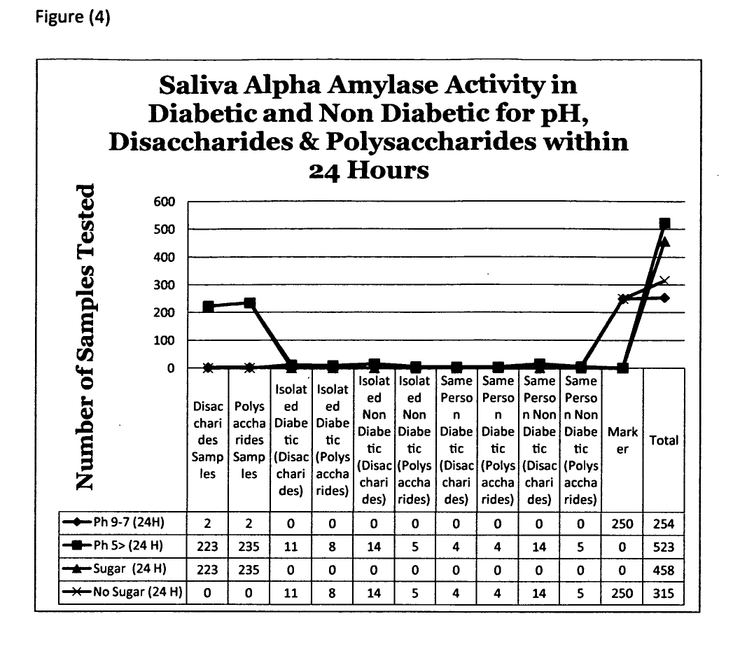

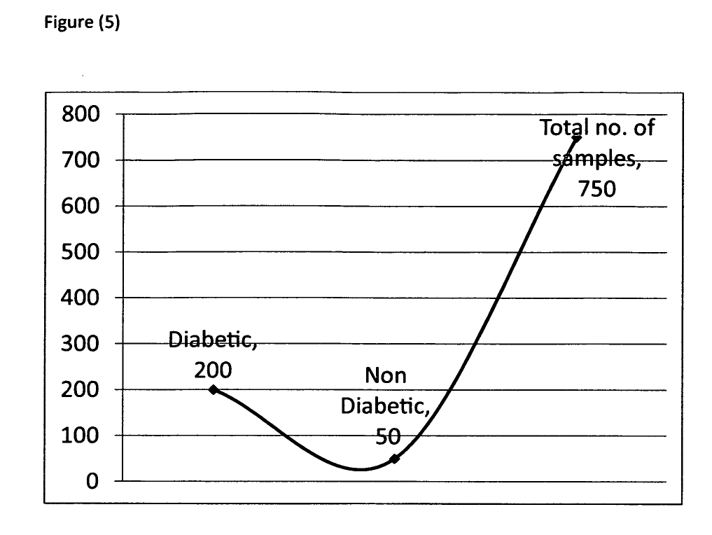

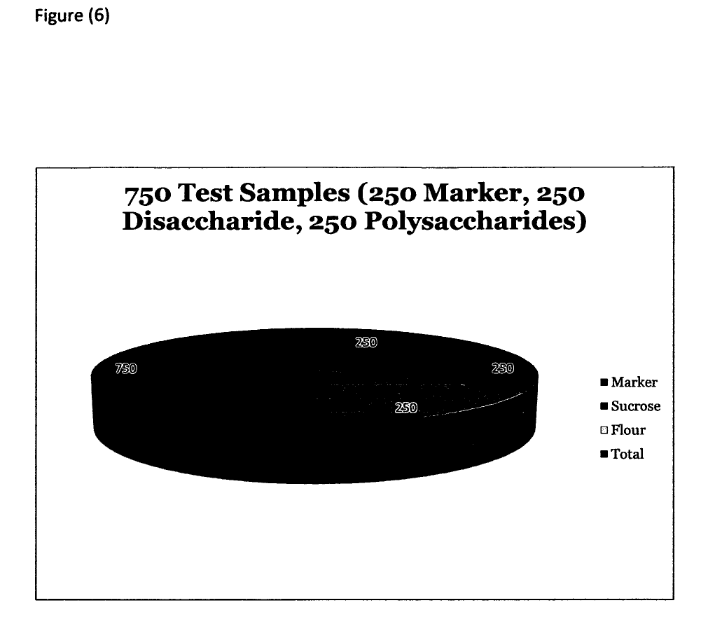

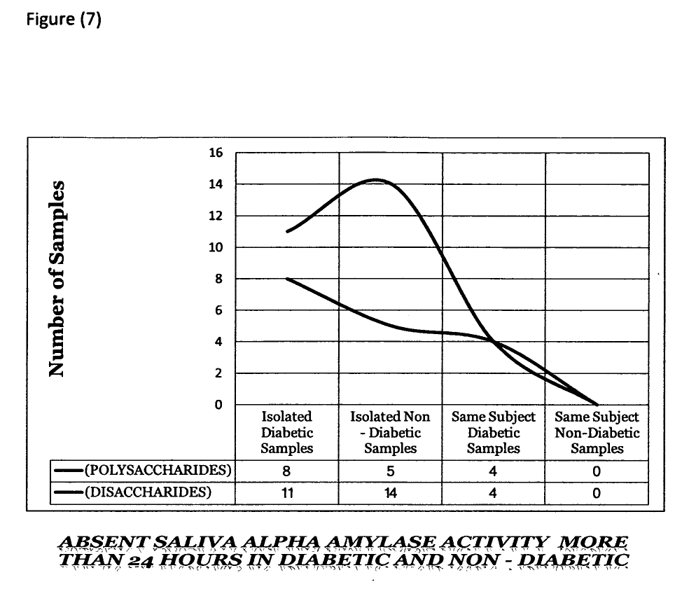

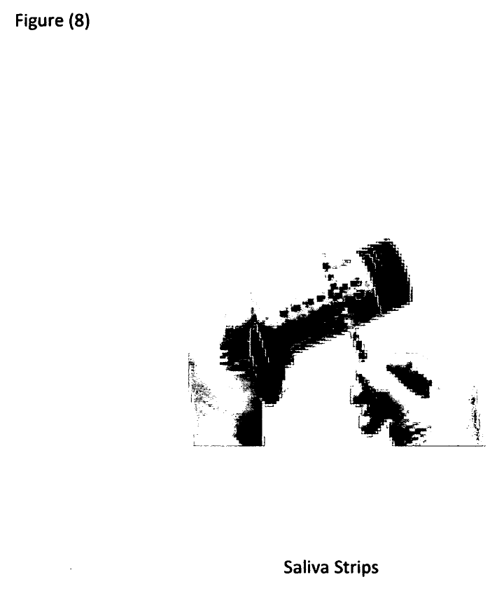

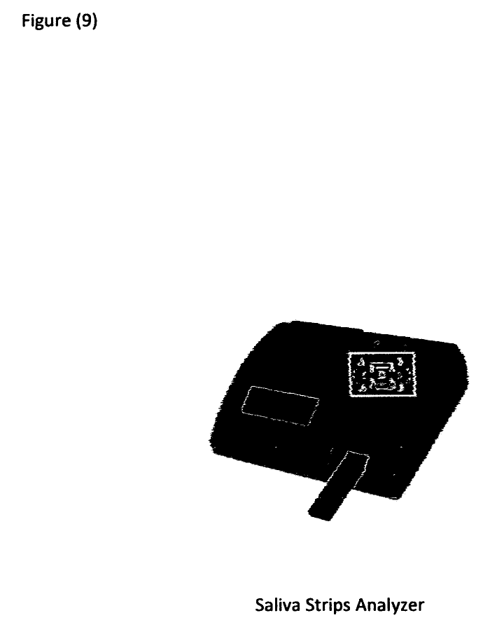

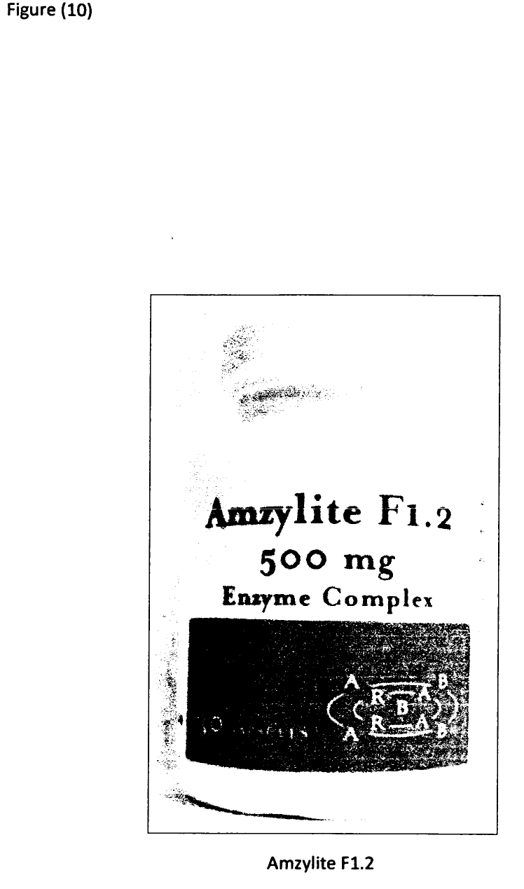

### CROSS REFERENCE TO RELATED APPLICATIONS

This application is a continuation of U.S. patent applications Ser. No. 12/135,179 filed Jun. 8, 2008 and Ser. No. 10/930,538, filed Apr. 30, 2004 designating the United States of America, the entire disclosure of which is incorporated herein by reference. Priority is claimed based on UK patent applications No. 0407583.4, filed May 4, 2004 and 0811274.9 filed Jun. 6, 2008.

### REFERENCES

Volkheimer G. [Persorption of microparticles] Pathologe 1993 September; 14(5):247-52. [Article in German]
Freedman B J. Persorption of raw starch: a cause of senile dementia? Med Hypotheses 1991 June; 35(2):85-7.
Volkheimer G. [The Herbst-Volkheimer effect]. [Article in German] Prokop O. Institut fur Gerichtliche Medizin des Bereichs Medizin (Charite) der Humboldt-Universitat zu Berlin, DDR. Kitasato. Arch Exp Med 1990 April; 63(I):I-6
Ullmann D, Connor W E, Hatcher L F, Connor S L, Flavell D P. Will a high-carbohydrate, low-fat diet lower plasma lipids and lipoproteins without producing hypertriglyceridemia? Department of Medicine, Oregon Health Sciences University, Portland 97201-3098. Arterioscler Thromb 1991 July-August; I 1(4): 1059-67
Liu GC1 Coulston A M, Reaven G M. Effect of high-carbohydrate-low fat diets on plasma glucose, insulin and lipid responses in hypertriglyceridemic humans. Metabolism 1983 August; 32(8):750-3 33.
Olefsky J M, Crapo P, Reaven G M. Postprandial plasma triglyceride and cholesterol responses to a low-fat meal. Am J Clin Nutr 1976 May; 29(5):535-9.
Ginsberg H, Olefsky J M, Kimmerling G, Crapo P, Reaven G M. J Clin Induction of hypertriglyceridemia by a low-fat diet. Endocrinol Metab 1976 April; 42(4):729-35

### BACKGROUND OF THE INVENTION

The present invention relates to the use of physiologically acceptable enzyme complex with active amylases, active lipases, active proteases, and specific proteins such as betaine, but especially of complex of digestive enzymes such as 1,4-α-D-glucan glucanohydrolase; Exo-1,4-α-glucosidase; Beta-fructofuranosidase; Protease; Pectinase; Lipase; Cellulase; Lactase; Malt Diastase, for the treatment of malfunctioning or absent amylases activity accompanied by/in relation to excess saccharides (polysaccharides, disaccharides and monosaccharides in the blood and body tissues) and for the manufacturing of medicinal products suitable for this treatment. The invention relates especially to the use of these enzyme complexes with active amylases, active lipases and active proteases, but especially of Betaine HCL; 1,4-α-D-glucan glucanohydrolase; Exo-1,4-α-glucosidase; Beta-fructofuranosidase; Protease; Pectinase; Lipase; Cellulase; Lactase; Malt Diastase, or complex of digestive enzymes containing Betaine HCL; 1,4-α-D-glucan glucanohydrolase; Exo-1,4-α-glucosidase; Beta-fructofuranosidase; Protease; Pectinase; Lipase; Cellulase; Lactase; Malt Diastase, for the adjuvant therapy of malfunctioning or absence of amylases activity in association with/presence of high levels of monosaccharides, disaccharides and polysaccharides in blood and tissues.

As used herein the term “presence of high levels of saccharides (polysaccharides, disaccharides and monosaccharides), in blood and tissues” is understood to mean a condition in which amylases activity is altered or impaired (inactivity of amylases enzymes for 24 hours or more) with or without, the presence of high levels of monosaccharides disaccharides and polysaccharides in blood and tissues in asymptomatic healthy individuals and symptomatic unhealthy individuals for example individuals suffer from renal failure.

Polysaccharides and disaccharides based diseases, the so-called “Saccharides sickness”. In addition to other, e.g. secondary forms of saccharides based diseases that can occur as sequelae of other primary diseases, many groups of disorders of carbohydrate metabolism are distinguished, i.e. Asthma, kidney failure, chronic wounds and ulcers, severe infections such as wet gangrene (gas gangrene), peripheral ischaemia, macro and microangiopthy, arthritis, D.V.T (deep vein thrombosis), P.E (pulmonary embolism), type I diabetes due to insulin deficiency and type II diabetes due to reduced insulin effectiveness, the course of the disease depending on the type concerned, among other factors. Peripheral ischaemia, is furthermore a chronic disease with a variety of pathological manifestations and is accompanied, for example, by disorders of lipid metabolism, circulation and saccharides metabolism. The typical symptoms of this disease include elevated inflammatory markers, deposit of polysaccharides and atheroma in blood vessels, vascular lumen and walls which could result in narrowing of vessels, development of skin wounds and chronic ulcers, tendency to infections and pruritus, and finally amputation of the limb. Peripheral ischaemia tends to be a progressive disorder and in many cases is also accompanied by various complications. Known complications include, for example, neurologic, chronic wounds, bone infections and vascular diseases. It is therefore necessary to adjust the therapy to meet each patient's individual requirements in every phase of the illness and to select the suitable medicinal product for each individual case. It may also be desirable for this therapy to supplement the selected primary medications with other medicinal products in the form of an adjuvant treatment which can exert a supporting effect on the therapy and beneficially influence the further course of the illness.

### SUMMARY OF THE INVENTION

Therefore, it is an object of the invention to provide a new method for the treatment of malfunctioning or absence of amylases activity in association with excess disaccharides and polysaccharides in blood and tissues in asymptomatic healthy individuals (as prophylaxis) and symptomatic unhealthy individuals.

It is another object of the invention to provide new pharmaceutical preparations for the treatment of malfunctioning or absence of amylases activity in association with excess disaccharides and polysaccharides in blood and tissues in asymptomatic healthy individuals and symptomatic unhealthy individuals.

It is a particular object of the invention to provide new pharmaceutical preparations for adjuvant therapy in the treatment of malfunctioning or absence of amylases activity in association with excess disaccharides and polysaccharides in blood and tissues in asymptomatic healthy individuals and symptomatic unhealthy individuals which exert an additional supportive effect on the treatment and beneficially influence the further course of the malfunctioning or absence of amylases activity in association with excess disaccharides and polysaccharides in blood and tissues, illness, for example by reducing the incidence of late complications.

According to the invention, physiologically acceptable enzyme mixtures with active amylase, active lipase and active protease, such as suitable enzyme mixtures of microbial origin and/or especially mixtures of digestive enzymes and proteins of plant origin such as preferably Betaine HCL; 1,4-α-D-glucan glucanohydrolase; Exo-1,4-α-glucosidase; Beta-fructofuranosidase; Protease; Pectinase; Lipase; Cellulase; Lactase; Malt Diastase, or Betaine HCL; 1,4-α-D-glucan glucanohydrolase; Exo-1,4-α-glucosidase; Beta-fructofuranosidase; Protease; Pectinase; Lipase; Cellulase; Lactase; Malt Diastase, —like mixtures of digestive enzymes, are used for the treatment of malfunctioning or absence of amylases activity in association with excess disaccharides and polysaccharides in blood and tissues in asymptomatic healthy individuals and symptomatic unhealthy individuals in larger mammals and humans, and for the manufacture of pharmaceutical preparations for such treatment.

For the present invention, physiologically acceptable enzyme mixtures with amylolytic, lipolytic, and proteolytic activity can be used that are of any human, plant or microbiological origin. The enzyme mixtures with active amylases, active lipases and active proteases used for the invention can be both of purely microbial origin, purely human origin, or may also be a complex of enzymes of human, plant and microbial origin.

In one variant of the invention, the enzyme mixture used is of purely microbial origin. Especially enzymes produced by bacteria, i.e. by the Bacillus or Pseudomonas strains, or by fungal cultures such as moulds, for example of the Rhizopus and Aspergillus strains, are especially suitable as microbial enzymes. Examples of such physiologically acceptable bacterial and/or mould fungi enzymes are already described in the state of the art, e.g. in connection with their synthesis and use for the treatment of digestive disorders. Lipases may be derived from, for example, Bacillus or Pseudomonas strains, amylases and lipases from mould fungi, for example of the Rhizopus strain, and proteases, for example, also from Aspergillus.

Another preferred variant of the invention, however, involves the use of complex of digestive enzymes and proteins with, Betaine HCL; 1,4-α-D-glucan glucanohydrolase; Exo-1,4-α-glucosidase; Beta-fructofuranosidase; Protease; Pectinase; Lipase; Cellulase; Lactase; Malt Diastase, that in their properties closely resemble gastric, pancreatic and intestinal enzymes. For the present invention, complex of digestive enzymes and proteins containing Betaine; 1,4-α-D-glucan glucanohydrolase; Exo-1,4-α-glucosidase; Beta-fructofuranosidase; Protease; Pectinase; Lipase; Cellulase; Lactase; Malt Diastase, and especially salivary, gastric, pancreatic and intestinal enzymes itself are preferably used, and one or more microbial enzymes, i.e. enzymes synthesized by microorganisms, of the group of amylases lipases, and proteases may if desired be added to the salivary, gastric, pancreatic and intestinal enzymes or the complex of digestive enzymes containing Betaine HCL; 1,4-α-D-glucan glucanohydrolase; Exo-1,4-α-glucosidase; Beta-fructofuranosidase; Protease; Pectinase; Lipase; Cellulase; Lactase; Malt Diastase.

Salivary like, gastric like, pancreatic like and intestinal like enzymes are known enzymes complex with active amylases, active lipases and active proteases which is available for example, under the trade name Amzylite®, in the form of granules, pellets or capsules containing enteric coated microspheres and is used medically for enzyme replacement, for example in digestive insufficiency after stomach operations, liver and biliary diseases, cystic fibrosis and chronic pancreatitis. Amzylite is generally obtained as a mixture of natural enzymes by extraction from microbial, plant, yeast, fungi and human saliva or pancreatic juice, for example according to the process described in UK patent application GB0407583.4, and is then converted into the desired galenical form in a manner known in the art. The salivary, gastric, pancreatic and intestinal like enzymes are usually administered orally or intravenously, in the form of solid or solution preparations.

In one variant of the invention, the pharmaceutical preparations manufactured and/or used in accordance with the invention contain preferably active amylases, active lipases and active proteases or complex of digestive enzymes and proteins containing Betaine HCL; 1,4-α-D-glucan glucanohydrolase; Exo-1,4-α-glucosidase; Beta-fructofuranosidase; Protease; Pectinase; Lipase; Cellulase; Lactase; Malt Diastase. These pharmaceutical preparations manufactured according to the invention can contain active amylases, active lipases and active proteases or complex of digestive enzymes and specific proteins containing Betaine HCL; 1,4-α-D-glucan glucanohydrolase; Exo-1,4-α-glucosidase; Beta-fructofuranosidase; Protease; Pectinase; Lipase; Cellulase; Lactase; Malt Diastase, and if desired in addition to active amylases one or more physiologically acceptable enzymes from the group of lipases, proteases, Betaine HCL; Beta-fructofuranosidase; Protease; Pectinase; Lipase; Cellulase; Lactase; Malt Diastase, of the kind that can be obtained from microorganisms. Microbial enzymes suitable for use as this supplement include especially the bacterially synthesized enzymes already mentioned above, for example by the Bacillus or Pseudomonas strains, or by fungal cultures such as mould fungi, for example of the Rhizopus or Aspergillus strains.

It has now been found that the physiologically acceptable enzyme complexes and specific proteins with Betaine; 1,4-α-D-glucan glucanohydrolase; Exo-1,4-α-glucosidase; Beta-fructofuranosidase; Protease; Pectinase; Lipase; Cellulase; Lactase; Malt Diastase, which can be obtained from microbial and/or human sources and described with reference to this invention, can be used not only for the treatment of digestive enzyme deficiency states—of the kind associated for example with pathological changes of the pancreas due to chronic pancreatitis, digestive insufficiency after stomach operations, liver or biliary diseases—but surprisingly are also suitable for the treatment of Asthma, kidney failure, chronic wounds and ulcers, severe infections such as wet gangrene (gas gangrene), peripheral ischaemia, macro and micro angiopthy, neuropathy, arthritis, D.V.T (deep vein thrombosis), P.E (pulmonary embolism), type I diabetes due to insulin deficiency and type II diabetes due to reduced insulin effectiveness, the course of the disease depending on the type concerned, among other factors in larger mammals and humans. In particular, the aforementioned physiologically acceptable enzyme complexes with active amylases, active lipases and active proteases, preferably however, for example, Amzylite itself or complexes of digestive enzymes containing 1,4-α-D-glucan glucanohydrolase; Exo-1,4-α-glucosidase; Beta-fructofuranosidase; Protease; Pectinase; Lipase; Cellulase; Lactase; Malt Diastase, which also contain microbial enzyme supplements, such as one or more microbial lipases, proteases and/or amylases, are suitable for adjuvant therapy in the management of saccharides based diseases, exert an additional effect supporting the saccharides based diseases therapy and beneficially influence the further course of the Asthma, kidney failure, chronic wounds and ulcers, severe infections such as wet gangrene (gas gangrene), peripheral ischaemia, macro and microangiopthy, neuropathy, arthritis, D.V.T (deep vein thrombosis), P.E (pulmonary embolism), diabetes, the course of the disease depending on the type concerned illness, i.e. especially by helping reduce the late complications of the saccharides based diseases associated with malfunctioning or absence of amylases activity disorders.

The use of the physiologically acceptable enzyme complexes, preferably, for example, Amzylite or complexes of digestive enzymes and specific proteins containing 1,4-α-D-glucan glucanohydrolase; Exo-1,4-α-glucosidase; Beta-fructofuranosidase; Protease; Pectinase; Lipase; Cellulase; Lactase; Malt Diastase, where appropriate with further microbial enzyme supplements in accordance with the invention also exhibits advantages in patients who in addition to the saccharides based diseases associated with malfunctioning or absence of amylases activity disorders have complications relating to coexisting salivary glands diseases, digestive system diseases and exocrine pancreatic insufficiency.

The pharmaceutical preparations manufactured and/or used according to the invention can contain in addition to the described microbial enzyme mixtures and/or mixtures of digestive enzymes of plant origin, such as especially 1,4-α-D-glucan glucanohydrolase; Exo-1,4-α-glucosidase; Beta-fructofuranosidase; Protease; Pectinase; Lipase; Cellulase; Lactase; Malt Diastase, or complexes of digestive enzymes containing salivary, gastric, pancreatic and intestinal enzymes, pharmaceutical excipients and/or additives and, if appropriate, stabilizers.

For example, the enzyme mixtures may be contained in an effective quantity (in each case determined on the basis of the active units of the active amylases, active lipases and active proteases components) together with conventional pharmaceutical excipients and/or vehicles in solid or liquid pharmaceutical preparations. The active amylases per dose unit may be generally within the range of 50 to 500 mg amylase units, active lipase per dose unit may be generally within a range of 10 to 50 mg lipase units, the active protease per dose unit may be generally within a range of 5 to 70 mg protease units. Examples of typical dose units are, for example, enzyme complex with the following activities: a) approx. 15 mg lipase/approx. 50 mg amylase/approx. 5 mg protease; b) approx. 50 mg lipase/approx. 100 mg amylase/approx. 25 mg protease; and c) approx. 25 mg lipase/approx. 150 mg amylase/approx. 60 mg protease units. Examples of solid formulations are preparations such as tablets, coated tablets, capsules, powder, granules or pellets for oral administration or preparations for intravenous administration. These solid preparations may contain conventional inorganic and/or organic pharmaceutical vehicles such as lactose, beet roots, dicalcium phosphate, and magnesium stearate, talc or starch as well as conventional pharmaceutical excipients such as glidants or tablet disintegrants. Liquid preparations such as solutions, suspensions or emulsions of the active ingredients may contain the usual diluents such as water, oils and/or suspending agents such as polyethylene glycols and the like. Further excipients such as preservatives, flavouring agents, stabilizers (e.g. complex lipids) and the like may also be added.

The enzyme complexes can be mixed and formulated with the pharmaceutical excipients and/or vehicles in a manner known to the art. To manufacture solid dosage forms, the enzyme complexes may, for example, be mixed with the excipients and/or vehicles in the usual manner and wet or dry granulated or pelleted. Granules, pellets or powder can be filled directly into capsules or sachets or compressed into tablet cores, or vial for intravenous administration in the usual manner. If desired, these cores can be coated to a dragee in the manner known to the art. Liquid preparations can be obtained by dissolving or dispersing the components and, if required, further excipients, in a suitable liquid vehicle, in the form of solutions or suspensions.

The anti-saccharides based diseases associated with malfunctioning or absence of amylases activity disorders and its complications, such as coexisting salivary glands diseases, digestive system diseases, peripheral vascular ischaemic diseases and exocrine pancreatic insufficiency effects and the beneficial influence on the course of the saccharides based diseases associated with malfunctioning or absence of amylases activity disorders and its complications illness, especially in adjuvant therapy as part of an saccharides based diseases regimen, can be demonstrated for the physiologically acceptable enzyme mixtures with active amylases, active lipases and active proteases used in accordance with the invention, such as, for example, the microbial enzyme mixtures and especially Amzylite or complexes of digestive enzymes and specific proteins containing 1,4-α-D-glucan glucanohydrolase; Exo-1,4-α-glucosidase; Beta-fructofuranosidase; Protease; Pectinase; Lipase; Cellulase; Lactase; Malt Diastase, described above, by determining the effect on pharmacological parameters of the kind generally used to evaluate saccharides and enzymes deficiency diseases. Such parameters may, for example, be an improvement, i.e. Decrease in saccharides level, improvement of enzymes activity and levels, improvement of kidney function tests such as urea and electrolytes, improvement of cholesterol levels and decrease in glycosylated hemoglobin (HbA_{1C}).

An in-vitro and in-vivo, single centre, non placebo-controlled, parallel group study with 1,4-α-D-glucan glucanohydrolase; Exo-1,4-α-glucosidase; Beta-fructofuranosidase; Protease; Pectinase; Lipase; Cellulase; Lactase; Malt Diastase, in the form of Amzylite® (capsules) was conducted in saccharides based diseases associated with malfunctioning or absence of amylases activity disorders and its complications patients with coexisting salivary malfunctioning and absence of salivary alpha amylase in association with excess presence of disaccharides and polysaccharides (saccharides) in the blood and tissues, to demonstrate the positive effects of the described enzyme complexes on diseases control. The patient sample (male/female) was randomly assigned to groups each comprising 141 patients. Treatment was given over a period of one year at an oral dosage of 3 to 6 Amzylite capsules daily. One or two capsules were administered before each of the main meals (3 to 4 daily). One capsule of Amzylite® contained medicinal ingredients: Betaine 200 mg; 1,4-α-D-glucan glucanohydrolase 100 mg; Exo-1,4-α-glucosidase 100 mg; Beta-fructofuranosidase 20 mg; Protease (3.0), 52.62 mg; Pectinase 10 mg; Lipase 7.5 mg; Cellulase 5 mg; Lactase 5 mg; Malt Diastase 3.33 mg. Non-Medicinal Ingredients: Microcrystalline cellulose, Magnesium stearate and maltodextrin. Parameters of the kind generally used to evaluate saccharides and enzymes deficiency diseases were recorded before start of treatment.

The efficacy of the enzyme complex used in terms of the salivary enzymes activity control was determined by measuring, saliva enzymes functions for disaccharides and polysaccharides, serum amylase levels, urea and electrolytes, cholesterol levels, saccharides levels in blood and body tissues, the glycosylated hemoglobin level (HbA_{1C}). In this context a positive influencing of the saliva enzymes function tests for saccharides, urea and electrolytes, cholesterol levels, saccharides levels in the blood and tissues and HbA_{1C} level as a clinically relevant improvement in the saccharides based diseases status is desired. Further saccharides based diseases parameters used were blood glucose levels (insulin/glucagon), status of lipid soluble vitamins (A, D and E), daily insulin dose, body weight index and hyperglycaemic episodes.

Following a preliminary assessment for selection of the patient sample, the patients completed an initial start-up phase of 1 to 2 weeks (without administration of enzyme complexes) to establish the patients on individual saccharides based diseases parameters before starting treatment with Amzylite. Before commencing the parallel groups study period, a baseline assessment was carried out. The patients underwent two interim assessments in the first month of the study. Further assessments were carried out after six weeks of treatment, then every 8 to 10 weeks and at the end of the study. Gastroenterologic parameters such as faecal fat, stool characteristics, coefficient of fat absorption (CFA) and clinical symptoms were evaluated separately from the saccharides based diseases and diabetic parameters.

The above study showed that saccharides based diseases in association with amylases malfunction or absent activity parameters are beneficially influenced by the administration of Amzylite, both in terms of a decrease of saccharides levels in blood and tissues, and improvement of amylases enzymes activity as well as improved symptoms of the diseases manifested, for example, by stabilization of blood saccharides and amylases activity curves and, for example, reduction of the symptoms and progress of the diseases. The results of the study therefore demonstrate that the administration of physiologically acceptable enzyme complexes with active amylases, active lipases and active proteases in addition to other saccharides related enzymes according to the invention can bring about an improvement in saccharides based diseases and amylases enzymes malfunction or absent activity and diabetes mellitus status. It was demonstrated independently of these findings that in the group of patients with amylases enzymes absent activity with coexisting complications, including diabetes the gastroenterologic parameters such as faecal fat, stool characteristics, CFA and clinical symptoms are also beneficially influenced and that the overall nutritional status of these patients is improved. Physiologically acceptable enzyme complexes with active enzymes such as 1,4-α-D-glucan glucanohydrolase; Exo-1,4-α-glucosidase; Beta-fructofuranosidase; Protease; Pectinase; Lipase; Cellulase; Lactase; Malt Diastase—especially Amzylite or complexes of enzymes containing 1,4-α-D-glucan glucanohydrolase; Exo-1,4-α-glucosidase; Beta-fructofuranosidase, also with the microbial enzyme supplements described—are therefore suitable for the manufacture of pharmaceutical preparations for the treatment of saccharides based diseases associated with or without malfunctioning amylases malfunctions or absent activity, especially for the adjuvant therapy of kidney failure, micro and macro angiopathy and diabetes mellitus; in these cases the diabetes mellitus may also be accompanied by amylases malfunction or absent activity in association with or without high levels of saccharides (mainly polysaccharides starch) in blood, blood vessels walls, organ tissues, skin and subcutaneous tissues and including brain tissues.

The pharmacological beneficial effects of the used enzyme complexes on the amylases malfunctioning or absent activity with or without saccharides based diseases and parameters and on the improvement of the saccharides based diseases, including diabetes mellitus status shall be further elucidated by the clinical study described in the following and by the results achieved therewith.

### Clinical Study

### Amzylite F 1.2 as In-vitro/In-vivo Glucose Reducing Agent and Treatment for Diabetes/Diabetes Complications

### Objectives:

Our objectives were to determine the efficacy of Amzylite as in-vitro/in-vivo hypoglycaemic agent, anti-diabetes anti-complications for both diabetes/non diabetes. Some of the studies were carried out at Medical & Health Center, Abu Dhabi, U.A.E (2006-2007) other studies were carried out at Buhaira Private Hospital-Omdurman-Sudan.

### Methods:

Our methods were: In-vitro testing to determine glucose units in a glucose solution before and after mixing with Amzylite against other known hypoglaycaemic agents (metformin, sulfonylurea, etc) including insulin. 10 mls of 10% concentrated glucose solution tested for glucose concentration using glucose oxidase and or hexokinase (glucometers can also be used). Glucose values were more than 600 mg/dl (Glucometer readings were Hi, 600 mg/dl or more;). One or two capsule of Amzylite was added to the glucose solution. In-vivo: 141 type I and type II diabetics with diabetes neuropathy, in addition to 9 diabetics with renal incompetency, 15 diabetics with chronic wounds to extremities, 2 diabetics had progressive gas gangrene, all patients had neuropathy with impaired sensations to distal extremities and one none diabetic with renal failure were identified. 31 out of 141 were excluded for incomplete records. 8 self discharged. 128 classified into 4 groups: 33 on insulin therapy; 20 on insulin and oral hypoglycaemic agents, 58 on only oral hypoglycaemic therapy, 16 on diet control only. 9 with high creatinine levels, 15 with chronic wounds, 2 had gas gangrene to distal extremities and one non diabetic had renal incompetency and was on dialysis. Laboratory tests were determined before and after starting Amzylite. No placebo was given.

### Results:

Our results showed In vitro glucose readings were significantly reduced immediately after mixing with Amzylite. Glucose values of 160 to 100 mg/dl and lower were obtained immediately. No changes observed with all other known hypoglycaemic agents (metformin, sulfonylurea, etc) including insulin. In vivo: 7 had diabetes reversed following treatment with Amzylite. 9 renal with high creatinine of up to 8 mg/dl (nr: 0.6-1.2 mg/dl) were recovered and condition reversed. One non diabetic dialysis patient with high cretinine of 14.5 mg/dl (nr: 0.6-1.2 mg/dl), reduced to 10.20 mg/dl within two month. 15 chronic wounds, reversed within 8 to 14 days. 2 gas gangrene (moist, wet gangrene), showed improvement, and disappearance of gas gangrene (disappearance of severe infection which resulted in stopping of progression of gangrene) by limitation of necrotic area but no change on necrotic tissues. One had very severe wound pains, required wound dressing under general anaesthesia. 128 reversed neuropathy and regained complete sensations. 89% managed to control symptoms and glucose readings without insulin or oral tablets. 10-11% managed to reduce insulin and tablets.

### Conclusion:

Amzylite should be considered for screening, prophylaxes and treatment of diabetes and its complications.

### Another Clinical Study

Amzylite F 1.2 as Glucose Reducing Agent with Direct Effect and Treatment for Diabetes.

### Introduction:

WHO defined diabetes as a chronic condition that occurs when the pancreas does not produce enough insulin or when the body cannot effectively use the insulin it produces.

### Objectives:

To determine the efficacy of Amzylite as in-vitro/in-vivo hypoglycaemic agent and anti-diabetic. To study the relationship between hypoglycaemic agents (including insulin) and glucose. Amzylite is a registered medicinal formulation.

### Settings:

In-vitro and in-vivo prospective studies were carried out at Medical & Health Center, Abu Dhabi, U.A.E.

### Methods:

In-vitro testing to determine glucose units in a glucose solution before and after mixing with Amzylite against other known hypoglaycaemic agents (metformin, sulfonylurea, etc) including insulin. 10 mls of 10% concentrated glucose solution tested for glucose concentration using glucose oxidase and or hexokinase (glucometers can also be used). Glucose values were more than 600 mg/dl (Glucometer readings were Hi, 600 mg/dl or more). One capsule of Amzylite added to the glucose solution. For In-vivo study to determine the effectiveness of Amzylite as treatment for D.M. 141 patients with type I and type II D.M were treated with Amzylite. 31 patients were excluded for incomplete records. 8 patients' self withdrawn. 102 Patients were classified into 4 groups: (1) 26 patients were on insulin therapy (4 of this group were excluded); (2) 9 were on insulin and oral hypoglycaemic agents, (2 were excluded) (3) 51 patients were on only oral hypoglycaemic therapy (4 were excluded from this group). (4) 16 patients were on diet control only (1 excluded from this group). Fasting and 2 hours postprandial blood glucose levels (HbA1c checked in some but not all patients) were determined before starting Amzylite. Clinical examinations together with clinical history were documented for each patient. Saliva enzymes function tests were determined for disaccharides and polysaccharides in all patients. Patients were requested to record 2 hour postprandial blood glucose values before and after Amzylite, 3 times per day or more. Improvement in patients' symptoms, daily glucose readings (fasting/2 hour postprandial) and HbA1c were set up as target and evaluation criteria. Follow up was set up every 2 to 4 weeks for daily glucose recording charts review, medication review, symptoms review and repeat saliva test after 4 weeks from the start of treatment. HbA1c test 2-3 months. No placebo was given. One or two capsules were given t.d.s or q.d.s, according to saliva enzymes function test result. Postprandial blood glucose tests were designed to determine and compare any changes in glucose values before and after Amzylite. Changes were also determined by 4 improvement criteria. 1—Improvement with complete recovery, 2—Significant improvement of symptoms and readings (range 70-170 mg/dl and below 200 mg/dl), 3—Improved symptoms but no change on readings, 4—Improved readings but no improvement on symptoms, 5—No change on symptoms or readings, 6—Higher or lower glucose readings. 7—Deterioration of symptoms.

### Results:

In vitro Results showed glucose readings were significantly reduced immediately after mixing with Amzylite. Glucose values of 180 mg/dl and lower were obtained immediately. Lower values were also obtained after 15, minutes and more (hours). No changes observed on glucose values when in vitro mixing glucose solution with all other known hypoglycaemic agents (metformin, sulfonylurea, etc) including insulin. In vivo results showed that, 7 out of 102 patients had completely recovered from diabetes within one, two and 3 months following treatment with Amzylite, (fasting and 2 hours postprandial blood glucose values were ranging 70-110 mg/dl). 88 patients had positive improvement on symptoms and readings (range 70-170 and below 200 mg/dl) within days to weeks following treatment with Amzylite. 3 patients improved on symptoms, without significant change on readings and 4 withdrawn. No patient had improved readings without improved symptoms. Zero patients had no change on symptoms or readings. Zero patients had very high or very low glucose readings. Zero patients had deterioration of symptoms. 89% of patients on Amzylite have managed to control their symptoms and glucose readings without insulin or oral hypoglaycaemic agents. 10-12% have managed to significantly reduce insulin (e.g. insulin reduced from 100 to 10 units per day) and tablets to one or half per day. Amzylite reacts directly with glucose to reduce it. Insulin and oral hypoglaycaemic agents react indirectly to reduce glucose by stimulating other enzymes (glucose reducing enzymes) which reduce glucose. Two thirds of fully recovered patients were newly diagnosed diabetics (diabetes for days, weeks and up to 6 months). Malfunction of any glucose reducing enzyme (e.g. Amylase) could result in diabetes, diabetes complication and insulin resistance.

### Conclusion:

Amzylite is a trade mark for a private formulation, registered in United Arab Emirates as dietary supplement. This study demonstrated that Amzylite F1.2 is an effective in-vitro and in-vivo glucose lowering agent as well as anti-diabetic agent. Study demonstrated that patients with diabetes can be reversed back to normal health, especially newly diagnosed. Diabetes screening programs should be considered in association with Amzylite for prevention or treatment of diabetes. Prophylaxes with Amzylite could help control spread of the disease. Amzylite capsules should be considered for treatment and prophylaxes against type 1 and type 2 diabetes mellitus. Due to its in-vitro immediate glucose lowering action Amzylite could become one of the most potential life saving hypoglycaemic agents.

### Another Clinical Study

### Diabetic and Non-Diabetic Saliva, Alpha Amylase Activity Increased in Acid Media

### Objectives:

To study pH changes during saliva alpha amylase activity in diabetic and non diabetic subjects in relation to glucose yielding. Explain mechanism of action and occurrence of DKA.

### Methods:

750 fresh saliva samples were collected from 200 diabetic and 50 non-diabetic subjects. All subjects were fasting over night for 12 hours. Glucose oxidase testing was used to detect amylase activity. Three identical 10 mls samples of fresh well-prepared saliva were accepted from each patient for the study. One sample from each subject was used as marker and tested for pH changes and sugar hourly for 24 hours. Each second and third study samples were tested initially for pH and sugar content; 1 ml of flour powder was added in each second sample (250 flour sample). 1 ml of sucrose was added in each third sample (250 sucrose samples). pH changes together with sugar readings were tested hourly in each study sample for 24 hours.

### Results:

750 (250 marker+250 flour+250 sucrose study sample) fresh saliva samples of non diabetics and diabetic patients showed pH of 9 to 7 on immediate testing. 250 marker samples showed no significant change in pH and zero sugar reading for 24 hours. 235 flour, 223 sucrose study samples showed decrease in pH 5>, in association with glucose detection in samples. 2 flour and 2 sucrose samples showed decrease in pH from 9 to 7 in association with glucose yielding within 24 hours. 8 isolated flour, 11 isolated sucrose diabetic samples, 4 same patient flour and sucrose diabetes samples, 5 flour and 14 sucrose non diabetic samples showed decrease in pH 5> without glucose yielding in 24 hours (absent amylase activity). Zero non diabetic samples, same subject showed absent amylase activity for both flour and sucrose. Decrease in pH in study samples was noticed to be associated with increase in glucose yielding. 92% of study samples showed decreased pH 5> in association with high glucose readings (250-500 mg/dl or more).

### Discussion:

Significant but nonspecific elevations of amylase can be seen in DKA (Diabetes Care 26:3193-3194, 2003). Similarity between DKA and this study in terms of increased acidity in the presence of elevated serum amylase, and in association with high glucose from degradation of disaccharides/polysaccharides particles that could exist in slightly larger amounts in the blood of diabetic compared to non diabetic subjects (persorption phenomenon-Gerhard Volkheimer).

### Conclusion:

Saliva alpha amylase activity should be determined in relation to pH media. Saliva alpha amylase is active in acid media. Correction of stomach pH and or abnormal amylase activity could help treat/prevent diabetes and obesity.

Overall, there is strong evidence that Amzylite is beneficial in the treatment of saccharides based diseases in association with malfunction or absence of amylases activity disorders and its complications patients with or without exocrine pancreatic insufficiency and leading to a better control of the disease. This means that Amzylite should be added to the treatment of those patients in order to improve the most important factor in their treatment in addition to asymptomatic patients as prophylaxis from the disease.

As kidney failure, peripheral ischaemic diseases and diabetic patients have been used as model, and it can be estimated that the same beneficial effects will be seen in other patients who are symptomatic or asymptomatic and suffer saccharides based diseases and its complications, as long as they would have a malfunctioning or absent amylases activity.

All patients involved in our studies gave informed consent.

See attached tables, graphs and figures used with the invention:

FIG. (1) Shows saliva amylase test results for disaccharides and polysaccharides together with pH and saliva proteins reaction. Glucose readings were taken every half an hour or every one hour. The glucose reading for disaccharides amylase reaction, pH and saliva proteins reaction is compared to glucose reading for polysaccharides amylase reaction, pH and saliva proteins reaction.

FIG. (2) Shows saliva amylase test results for disaccharides, and in the same time, the saliva sample is tested for leucocytes, protein, pH, blood, specific gravity, and glucose.

FIG. (3) Shows saliva amylase test results for polysaccharides, and in the same time, the saliva sample is tested for leucocytes, protein, pH, blood, specific gravity, and glucose.

FIG. (4) Saliva alpha amylase activity in diabetic and non diabetic subjects, pH, disaccharides and polysaccharides studies, within 24 hours.

FIG. (5) Total number of samples in relation to number of sources; samples were taken from diabetic and non diabetic subjects.

FIG. (6) 750 saliva samples studied for amylase activity, (250 saliva marker samples, 250 saliva sample for disaccharide testing, and 250 saliva samples for polysaccharides testing)

FIG. (7) Saliva alpha amylase activity study in diabetic and non diabetic subjects for more than 24 hours

FIG. (8) Saliva strips for determination glucose (Glucose oxidase based strips), bilirubin, specific gravity, blood, pH, protein, leucocytes, disaccharides and polysaccharides in saliva samples.

FIG. (9) Saliva strips Analyser for determination of glucose, bilirubin, specific gravity, blood, pH, protein, leucocytes, disaccharides, polysaccharides in saliva samples. Disaccharides reagents (Standard disaccharides reagent “sucrose”) and polysaccharides reagents (standard polysaccharides reagent “starch”) can be mixed in fresh saliva samples, pancreatic juice samples, urine samples to determine amylase activity for disaccharides and polysaccharides.

FIG. (10) AMZYLITE (Enzyme Complex): Each capsule contains: Medicinal Ingredients: Betaine; 1,4-α-D-glucan glucanohydrolase; Exo-1,4-α-glucosidase; Beta-fructofuranosidase; Protease (3.0); Pectinase; Lipase; Cellulase; Lactase; Malt Diastase. Non-Medicinal Ingredients: Microcrystalline cellulose, Magnesium stearate and maltodextrin.

The following example illustrates the manufacture of a pharmaceutical preparation containing Betaine; 1,4-α-D-glucan glucanohydrolase; Exo-1,4-α-glucosidase; Beta-fructofuranosidase; Protease; Pectinase; Lipase; Cellulase; Lactase; Malt Diastase, which is suitable for the treatment of malfunctioning or absence of amylase activity in association with saccharides based diseases, and especially for adjuvant therapy of kidney failure, and diabetes, without, however, restricting the scope of the invention.

### Examples of Pharmaceutical Formulation

One example of a pharmaceutical formulation includes a capsule containing: Betaine 400 mg; Amylase 100 mg; Maltase 10 mg; Sucarase 10 mg; Lactase 10 mg; and Cellulase 10 mg. Non-Medicinal Ingredients: Beet roots, Dicalcium phosphate, magnesium stearate.

Another example of a pharmaceutical formulation includes a capsule containing: Amylase 50 mg; Betaine 200 mg; Lipase 80 mg; Protease 100 mg; Maltase 20 mg; Lactase 20 mg; Cellulase 20 mg; and Sodium bicarbonate 0.5 mg. Non-Medicinal Ingredients: Beet roots, Dicalcium phosphate, magnesium stearate.

Another example of a pharmaceutical formulation includes a capsule containing: Betaine HCL 300 mg; 1,4-α-D-glucan glucanohydrolase 50 mg; Exo-1,4-α-glucosidase 50 mg; Beta-fructofuranosidase 10.0 mg; Maltase 10 mg; Lactase 10 mg; Cellulase 10 mg; Protease 40 mg; Papain 26 mg; Bromelain 26 mg; and Lipase 26 mg. Encapsulated with these ingredients: Beet roots, Dicalcium phosphate, magnesium stearate.

Another example of a pharmaceutical formulation includes a capsule containing: Betaine HCL 200 mg; 1,4-α-D-glucan glucanohydrolase 150 mg; Exo-1,4-α-glucosidase 150 mg; Beta-fructofuranosidase 20.0 mg; Maltase 10 mg; Lactase 10 mg; Cellulase 10 mg; Non-Medicinal Ingredients: Beet root, Magnesium stearate and maltodextrin.

Another example of a pharmaceutical formulation includes a capsule containing: 1,4-α-D-glucan glucanohydrolase 100 mg; Exo-1,4-α-glucosidase 100 mg; beta-fructofuranosidase 20 mg, Betaine 100 mg; Non-Medicinal Ingredients Microcrystalline cellulose, Magnesium stearate and maltodextrin.

Another of example of pharmaceutical formulation: Betaine HCL 300 mg; 1,4-α-D-glucan glucanohydrolase 100 mg; Exo-1,4-α-glucosidase 100 mg; Beta-fructofuranosidase 12.0 mg; Protease 40 mg; Papain 26 mg; Bromelain 26 mg; Lipase 26 mg; and Sodium bicarbonate 1.25 g. Encapsulated with these natural ingredients: Dicalcium phosphate, magnesium stearate.

Another of example of pharmaceutical formulation under the trade name of AMZYLITE (Enzyme Complex): Each capsule contains: Medicinal Ingredients: Betaine 200 mg; 1,4-α-D-glucan glucanohydrolase 100 mg; Exo-1,4-α-glucosidase 100 mg; Beta-fructofuranosidase 2.0 mg; Protease (3.0), 62 mg; Pectinase 10 mg; Lipase 7.5 mg; Cellulase 5 mg; Lactase 5 mg; Malt Diastase 3.33 mg. Non-Medicinal Ingredients: Microcrystalline cellulose, Magnesium stearate and maltodextrin.

Another example of a pharmaceutical formulation includes a capsule containing: Betaine 400 mg; 1,4-α-D-glucan glucanohydrolase 25 mg; Exo-1,4-α-glucosidase 100 mg; Beta-fructofuranosidase 10.0 mg; Maltase 10 mg; Lactase 10 mg; Cellulase 10 mg; Lipase 26 mg; Encapsulated with these ingredients: beet root, magnesium stearate.

Another example of a pharmaceutical formulation includes a capsule containing: 1,4-α-D-glucan glucanohydrolase 100 mg; Exo-1,4-α-glucosidase 100 mg; Beta-fructofuranosidase 15.0 mg; Betaine HCL 400 mg; Maltase 10 mg; Malt diastase 270 DP; Lactase 10 mg; Cellulase 10 mg; Protease 40 mg; Papain 26 mg; Pectinase (with phytase) 75 ENDQ/PGU; Bromelain 26 mg; Lipase 26 mg; Encapsulated with these ingredients: beet root, Dicalcium phosphate, magnesium stearate.

Another example of a pharmaceutical formulation includes a capsule containing: Betaine HCL 400 mg; 1,4-α-D-glucan glucanohydrolase 100 mg; Exo-1,4-α-glucosidase 100 mg; Beta-fructofuranosidase 10.0 mg; Encapsulated with these ingredients: beet root, magnesium stearate.

Another example of a pharmaceutical formulation includes a capsule containing: Betaine HCL 300 mg; 1,4-α-D-glucan glucanohydrolase 200 mg; Exo-1,4-α-glucosidase 100 mg; Beta-fructofuranosidase 20.0 mg; Encapsulated with these ingredients: beet root, magnesium stearate.

Another example of a pharmaceutical formulation includes a capsule containing: Betaine HCL 100 mg; 1,4-α-D-glucan glucanohydrolase 100 mg; Exo-1,4-α-glucosidase 100 mg; Beta-fructofuranosidase 15.0 mg; Encapsulated with these ingredients: beet root, magnesium stearate.

### Saliva, Urine, Blood, Lymphatic Fluid, Testing Strips and Testing Meters for Use with the Invention

New Glucose blood testing strips, and new glucometer (ARBAB): All modern hand-held “finger-stick” blood-glucose meters are based on the reaction between glucose and an enzyme that catalyzes the oxidation of glucose (usually the enzyme called, logically enough, “glucose oxidase”). In this reaction, glucose is oxidized to hydrogen peroxide plus gluconic acid. A newer generation of glucose meters incorporates an electrochemical cell within the device, and measures the integrated current produced by the glucose oxidation reaction, a quantity that is proportional to the amount of glucose present. Our new glucose meter (ARBAB-Glucometer), is using new technology based on the reaction between glucose and Amzylite or glucose and an organic compound called Trimethylglycine, also called TMG (Betaine HCL which is a chloride salt from TMG) that catalyzes the hydrolysis/oxidation of glucose. In this reaction, glucose is hydrolysed and oxidized to carbon dioxide and water, or to hydrogen and peroxide plus gluconic acid. A newer generation of glucose meters incorporates an electrochemical cell within the device, and measures the integrated current produced by the glucose oxidation reaction, a quantity that is proportional to the amount of glucose present. There are several key characteristics of “ARBAB-Glucometer”, glucose meters that may differ from model to model:

Size: There are different sizes, and the average size is now approximately the size of the palm of the hand, though some are smaller or larger. They are battery-powered.

Test strips: ARBAB-Glucometer tests strips are a consumable element containing enzymes and chemicals (Amzylite or Betaine HCL {Trimethylglycine, TMG}) that react with glucose in the drop of blood is used for each measurement. For some models this element is a plastic test strip with a small spot impregnated with Amzylite or Betaine HCL {Trimethylglycine, TMG} and other components. Each strip can only be used once and is then discarded. Instead of strips, some models use discs that may be used for several readings.

Volume of blood sample: The size of the drop of blood needed by different models varies from 0.3 to 10 μl. (Older models required larger blood samples, usually defined as a “hanging drop” from the fingertip.) Smaller volume requirements reduce the frequency of unproductive pricks.

Testing times: The times it takes to read a test strip may range from 3 to 30 seconds for different models.

Display: The glucose value in mg/dl or mmol/l is displayed in a small window. The preferred measurement unit varies by country: mg/dl is preferred in the US, mmol/l in Canada and Europe. To convert mmol/l of glucose to mg/dl, multiply by 18. To convert mg/dl of glucose to mmol/l, divide by 18 or multiply by 0.055.

Clock/memory: All ARBAB-Glucometers now include a clock that is set for date and time, and a memory for past test results. The memory is an important aspect of diabetes care, as it enables the person with diabetes to keep a record of management and look for trends and patterns in blood glucose levels over days. Most memory chips can display an average of recent glucose readings.

Data transfer: Some of ARBAB-Glucometers have more sophisticated data handling capabilities. Many can be downloaded by a cable or infrared to a computer that has diabetes management software to display the test results. Some meters allow entry of additional data throughout the day, such as insulin dose, Amzylite dose, amounts of carbohydrates eaten, or exercise.

Hospital glucose meters: Special glucose meters (ARBAB-Glucometer) for multi-patient hospital use will be available soon. These will provide more elaborate quality control records, and the data handling capabilities are designed to transfer glucoses into electronic medical records and the laboratory computer systems for billing purposes.

New urine glucose testing strips and new glucometer (ARBAB-Uro-Glucometer): Technique is similar to blood testing strips. Our new glucose meter (ARBAB-Uro-Glucometer), is using new technology based on the reaction between glucose and Amzylite or glucose and an organic compound called Trimethylglycine, also called TMG (Betaine HCL which is a chloride salt from TMG) that catalyzes the hydrolysis/oxidation of glucose. In this reaction, glucose is hydrolysed and oxidized to carbon dioxide and water, or to hydrogen and peroxide plus gluconic acid. A newer generation of glucose meters incorporates an electrochemical cell within the device, and measures the integrated current produced by the glucose oxidation reaction, a quantity that is proportional to the amount of glucose present. There are several key characteristics of “ARBAB-Uro-Glucometer”, glucose meters that may differ from model to model:

Size: There are different sizes, and the average size is now approximately the size of the palm of the hand, though some are smaller or larger. They are battery-powered.

Test strips: ARBAB-Uro-Glucometer tests strips are a consumable element containing enzymes and chemicals (Amzylite or Betaine HCL {Trimethylglycine, TMG}) that react with glucose in the drop of urine is used for each measurement. For some models this element is a plastic test strip with a small spot impregnated with Amzylite or Betaine {Trimethylglycine, TMG} and other components. Each strip can only be used once and is then discarded. Instead of strips, some models use discs that may be used for several readings.

Volume of blood sample: The size of the drop of urine needed by different models varies from 0.3 to 10 μl. Smaller volume requirements reduce the frequency of unproductive repeat samples.

Testing times: The times it takes to read a test strip may range from 3 to 30 seconds for different models.

Display: The glucose value in mg/dl or mmol/l is displayed in a small window. The preferred measurement unit varies by country: mg/dl is preferred in the US, mmol/l in Canada and Europe. To convert mmol/l of glucose to mg/dl, multiply by 18. To convert mg/dl of glucose to mmol/l, divide by 18 or multiply by 0.055.

Clock/memory: All ARBAB-Uro-Glucometers now include a clock that is set for date and time, and a memory for past test results. The memory is an important aspect of diabetes care, as it enables the person with diabetes to keep a record of management and look for trends and patterns in blood glucose levels over days. Most memory chips can display an average of recent glucose readings.

Data transfer: Some of ARBAB-Uro-Glucometers have more sophisticated data handling capabilities. Many can be downloaded by a cable or infrared to a computer that has diabetes management software to display the test results. Some meters allow entry of additional data throughout the day, such as insulin dose, Amzylite dose, amounts of carbohydrates eaten, or exercise.

Hospital glucose meters: Special glucose meters (ARBAB-Uro-Glucometer) for multi-patient hospital use will be available soon. These will provide more elaborate quality control records, and the data handling capabilities are designed to transfer glucoses into electronic medical records and the laboratory computer systems for billing purposes.

Urine strips (dip sticks): A testing dipstick is usually made of paper or cardboard and is impregnated with reagents that indicate some feature of the liquid by changing colour. For example, medical dipsticks are used to test urine samples for haemoglobin, nitrite (produced by bacteria in a urinary tract infection), protein, glucose and occasionally urobilinogen or ketones. It is used for determination glucose (using Amzylite or betaine as reagents on the strips), bilirubin, specific gravity, blood, pH, protein, leucocytes, in urine samples.

Saliva strips for determination, bilirubin, specific gravity, blood, pH, protein, leucocytes, and measuring saliva enzymes activity for disaccharides in saliva samples. Strips comprise of multi small paper or cardboard and each is impregnated with reagents that indicate some feature of the liquid by changing colour. For example, strips used are similar to medical dipsticks used to test urine samples for haemoglobin, nitrite (produced by bacteria in a urinary tract infection), protein, glucose and urobilinogen or ketones. To measure Amylase activity for disaccharides in a saliva sample, you will need 1 ml of organic disaccharide (sachets containing standard amount of disaccharides for the reaction together with manual of use and instructions will be supplied) to be mixed in 10 mls of fresh saliva, collected in a sterile specimen pot with lid. Readings will be taken immediately before adding disaccharides from sachets, then continuous reading to be obtained every half an hour or every hour for 24 hours. Results will be noted in a specially designed note book or graphs and or printed analysis paper from the strips analyzer machine. (See examples of test results note book used before in our Medical clinic in UAE and Sudan). You can use this test to measure saliva amylase activity for 1—Genetically Modified disaccharides, 2—Resistant disaccharides (sucrose from resistant plant source).

Saliva strips for determination, bilirubin, specific gravity, blood, pH, protein, leucocytes, and measuring saliva enzymes activity for polysaccharides in saliva samples. Strips comprise of multi small paper or cardboard and each is impregnated with reagents that indicate some feature of the liquid by changing colour. For example, strips used are similar to medical dipsticks used to test urine samples for haemoglobin, nitrite (produced by bacteria in a urinary tract infection), protein, glucose and urobilinogen or ketones. To measure Amylase activity for polysaccharides in a saliva sample, you will need 1 ml of organic polysaccharide starch (sachets containing standard amount of polysaccharides starch for the reaction together with manual of use and instructions will be supplied) to be mixed in 10 mls of fresh saliva, collected in a sterile specimen pot with lid. Readings will be taken immediately before adding polysaccharide starch, then continuous reading to be obtained every half an hour or every hour for 24 hours. Results will be noted in a specially designed note book or graphs and or printed analysis paper from the strips analyzer machine. (See examples of test results note book used before in our Medical clinic in UAE and Sudan). You can use this test to measure saliva amylase activity for 1—Genetically Modified polysaccharides starch, 2—Resistant polysaccharides from resistant plant source).

Saccharides Enzymes activity functions strips, to determine both the presence and the level of activity of 1,4-α-D-glucan glucanohydrolase; Exo-1,4-α-glucosidase; Beta-fructofuranosidase; Protease; Pectinase; Lipase; Cellulase; Lactase; Malt Diastase enzymes in venous blood samples, arterial blood samples, capillary blood samples, serum blood samples, lymphatic fluid samples. Strips contain, (1,4-α-D-glucan glucanohydrolase; Exo-1,4-α-glucosidase; Beta-fructofuranosidase; Protease; Pectinase; Lipase; Cellulase; Lactase; Malt Diastase). Strips will be read by special strip analyzer.

Saccharides Enzymes activity functions strips, to determine both the presence and the level of activity of 1,4-α-D-glucan glucanohydrolase; Exo-1,4-α-glucosidase; Beta-fructofuranosidase; Protease; Pectinase; Lipase; Cellulase; Lactase; Malt Diastase enzymes in venous blood samples, arterial blood samples, capillary blood samples, serum blood samples, lymphatic fluid samples, and urine samples. Strips contain, saccharides reagents (sachets with different types of saccharides in powder or liquid form for reaction with enzymes in the blood, urine, lymphatic fluid and saliva samples) containing polysaccharides starch, disaccharides (sucrose), dextrin, proteins, pectin, lipids, cellulose, lactose and maltose are to be added in saliva samples and urine samples, to determine presence and or level of activity for saccharides enzymes contained in a sample. Strips will be read by special strip analyzer.

### Strips Analyzer for Saliva, and Urine Strips

Urine strips Analyzer for determination glucose, bilirubin, specific gravity, blood, pH, protein, leucocytes, haemoglobin, nitrite (produced by bacteria in a urinary tract infection), urobilinogen and ketones. in urine samples. An analyzer for testing dipsticks impregnated with reagents that indicate some feature of the liquid by changing colour. For example, it is similar to medical dipsticks analyzer used to test urine samples for glucose, bilirubin, specific gravity, blood, pH, protein, leucocytes, haemoglobin, nitrite (produced by bacteria in a urinary tract infection), urobilinogen and ketones. Here dipstick uses Amzylite or betaine as reagents instead of glucose oxidase or dehydrogenase enzymes, on the strip to determine glucose in a urine sample.

Saliva strips Analyzer for determination Saliva strips for determination, of specific gravity, blood, pH, protein, leucocytes, and measuring saliva enzymes activity for polysaccharides in saliva samples. Strips comprise of multi small paper or cardboard and each is impregnated with reagents that indicate some feature of the liquid by changing colour. For example, strips used are similar to medical dipsticks used to test urine samples for haemoglobin, nitrite (produced by bacteria in a urinary tract infection), protein, glucose and urobilinogen or ketones. To measure Amylase activity for polysaccharides in a saliva sample, you will need 1 ml of organic polysaccharide starch (sachets containing standard amount of polysaccharides starch for the reaction together with manual of use and instructions will be supplied) to be mixed in 10 mls of fresh saliva, collected in a sterile specimen pot with lid. Readings will be taken immediately before adding polysaccharide starch, then continuous reading to be obtained every half an hour or every hour for 24 hours. Results will be noted in a specially designed note book or graphs and or printed analysis paper from the strips analyzer machine. (See examples of test results note book used before in our Medical clinic in UAE and Sudan). You can use this test to measure saliva amylase activity for 1—Genetically Modified polysaccharides starch, 2—Resistant polysaccharides from resistant plant source). Disaccharides reagents (Standard disaccharides reagent “sucrose”) and polysaccharides reagents (standard polysaccharides reagent “organic starch”) can be mixed in separate samples of fresh saliva samples, pancreatic juice samples, urine samples to determine amylase activity for disaccharides and polysaccharides.

### Multifunction-Urine Strips Analyzer

### Detailed Product Description

### Specifications:

10, Items to be tested: Leukocytes, Occult Blood, Nitrite, Urobilinogen, Protein, pH, Specific Gravity, Ketone, Bilirubin, Glucose.

11, Items to be tested: Leukocytes, Occult Blood, Nitrite, Urobilinogen, Protein, pH, Specific Gravity, Ketone, Bilirubin, Glucose, Calcium.

Strips are using new technology based on the reaction between glucose and Amzylite or glucose and an organic compound called Trimethylglycine, also called TMG (Betaine which is a chloride salt from TMG) that catalyzes the hydrolysis/oxidation of glucose. In this reaction, glucose is hydrolysed and oxidized to carbon dioxide and water, or to hydrogen and peroxide plus gluconic acid. Test theory: Test by cold light; Wavelength: Measurement wavelengths: 420 nm, 550 nm, 650 nm; Print: Built in speedy thermal printer outlet to pin printer; Methods: Test strip colour change by three wavelength; Test speed: 60 specimens per hour; Data output: USB Port to PC; Display: Liquid crystal; Operating environment: Temperature 10˜35 humidity≦95%; Power: AC220±22V, 50±1 HZ, or DC9V, 1.5 A 12 VA.

Normal Ranges for Saliva Amylase activity on Disaccharides (Standard organic, non GM, non resistant, Sucrose reagent) and Saliva Amylase activity on Polysaccharides (Standard organic, non GM, non resistant, polysaccharides starch) tests results

According to the invention, normal ranges of amylases activity are: 30 to 120 minutes for the degradation of disaccharides by the disaccharides specialised amylases (disaccharides specialised salivary, pancreatic or systemic amylases were not known before), and 4 to 6, up to 8 hours for the degradation of polysaccharides by polysaccharides specialised amylases. Tests results for amylase activity on polysaccharides, and amylase activity on disaccharides is compared to a normal range of 30 to 120 minutes for disaccharides, and 6 to 8 hours for polysaccharides (meaning: 250 mg/dl of glucose detected in a disaccharide test sample within 30 to 120 minutes, and 250 mg/dl of glucose to be detected in a polysaccharides sample within 6 to 8 hours). Normal range for disaccharides and polysaccharides are the ranges detected in a healthy non polysaccharides, oligosaccharides, disaccharides and monosaccharides diseases, non diabetic, non diseased individuals in a low endemic diabetes area (normal range figures obtained from health subjects in western Sudan and Darfur region where diabetes is approximately 7% of the population). Normal ranges for the tests can also be compared from the following calculations: Glucamylase: 1 unit of enzyme activity catalyzes the production of 1.0 mg (1.0 ml) of glucose in 1 hour under the following condition: 40 C pH=4.6, Amylase: 1 unit of enzyme activity is the amount of enzyme that will dextrinize 1.0 mg (1.0 ml) of soluble starch in 1 hour under the following condition: 60 C pH=6.0, High temperature amylase: 1 unit of enzyme activity is the amount of enzyme that will dextrinize 1.0 mg (1.0 ml) of soluble starch in 1 hour under the following condition: 70 C pH=6.0.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations falling within the scope of the appended claims and equivalents thereof.

What is claimed is:

1. A method of treating a larger mammal or human suffering from primary malfunctioning or absence activity of amylases in saliva samples, pancreatic juice samples or urine samples, comprising administering to said mammal or human an effective amount of a pharmaceutical preparation comprising a physiologically acceptable enzyme complex having active amylases, active lipases, active proteases (for example: 4-α-D-glucan glucanohydrolase; Exo-1,4-α-glucosidase; Beta-fructofuranosidase; Protease (3.0); Pectinase; Lipase; Cellulase; Lactase; Malt Diastase).

2. A method of treating a larger mammal or human suffering from primary high levels/excess or deposit of saccharides (polysaccharides and disaccharides) in the blood, urine, lymphatic fluid and tissues (for example subcutaneous tissue), comprising administering to said mammal or human an effective amount of a pharmaceutical preparation comprising a physiologically acceptable enzyme complex having active amylases, active lipases, active proteases (for example: 4-α-D-glucan glucanohydrolase; Exo-1,4-α-glucosidase; Beta-fructofuranosidase; Protease (3.0); Pectinase; Lipase; Cellulase; Lactase; Malt Diastase).

3. A method according to claim 1, wherein said pharmaceutical preparation comprises a physiologically acceptable enzyme complex of human salivary, pancreatic amylases, or plant and or microbially synthesized lipases, proteases and amylases.

4. A method according to claim 1, wherein said pharmaceutical preparation comprises 1,4-α-D-glucan glucanohydrolase; Exo-1,4-α-glucosidase; Beta-fructofuranosidase; Protease (3.0); Pectinase; Lipase; Cellulase; Lactase; Malt Diastase.

5. A method according to claim 1, wherein said pharmaceutical preparation comprises a complex of digestive enzymes containing, 1,4-α-D-glucan glucanohydrolase; Exo-1,4-α-glucosidase; Beta-fructofuranosidase; Protease; Pectinase; Lipase; Cellulase; Lactase; Malt Diastase.

6. A method according to claim 3, wherein said pharmaceutical preparation comprises 1,4-α-D-glucan glucanohydrolase; Exo-1,4-α-glucosidase; Beta-fructofuranosidase; Protease; Pectinase; Lipase; Cellulase; Lactase; Malt Diastase
or a complex of digestive enzymes containing Betaine HCL; 1,4-α-D-glucan glucanohydrolase; Exo-1,4-α-glucosidase; Beta-fructofuranosidase; Protease; Pectinase; n
Lipase; Cellulase; Lactase; Malt Diastase, and at least one microbial enzyme selected from the group consisting of lipases, proteases and amylases.

7. A method according to claim 1, wherein said pharmaceutical preparation is administered as an adjuvant therapy in conjunction with another, primary therapy.

8. A method according to claim 1, wherein said larger mammal or human is a patient suffering from malfunctioning or absent amylase activity for 24 hours or more accompanied or unaccompanied with high levels of saccharides (polysaccharides, disaccharides, oligosaccharides and monosaccharides) in the blood, urine or body tissues for example subcutaneous tissue.

9. New glucose test strips using new technology based on the reaction between glucose and Amzylite or glucose and an organic compound called Trimethylglycine, also called TMG or Betaine.
